(19) United States
(12) Patent Application Publication
(10) Pub. No.: US 2025/0270591 A1
(43) Pub. Date: Aug. 28, 2025

(54) NANOWIRE-BASED INOCULATION SYSTEM FOR DELIVERING GENETIC MATERIAL INTO SINGLE CELL AND FABRICATION METHOD THEREOF

(71) Applicant: POSTECH RESEARCH AND BUSINESS DEVELOPMENT FOUNDATION, Pohang-si (KR)

(72) Inventors: Seung Soo OH, Pohang-si (KR); Jung Ho JE, Pohang-si (KR); Un Yang, Pohang-si (KR); Byung Hwa Kang, Pohang-si (KR); Seol Yeong Jeong, Pohang-si (KR)

(73) Assignee: POSTECH RESEARCH AND BUSINESS DEVELOPMENT FOUNDATION, Pohang-si (KR)

(21) Appl. No.: 18/912,695

(22) Filed: Oct. 11, 2024

(30) Foreign Application Priority Data
Feb. 27, 2024 (KR) 10-2024-0027987

(51) Int. Cl. C12N 15/87; C12N 13/00

(52) U.S. Cl. CPC C12N 15/87; C12N 13/00

(57) ABSTRACT

The present invention relates to a nanowire-based inoculation system for delivering genetic material into a single living cell and a method for fabricating the same. Specifically, the present invention relates to a nanowire-based inoculation system capable of delivering genetic material into a desired location in a single cell through evanescent field-driven release of the genetic material from the nanowire surface. The nanowire-based inoculation system for delivering genetic material into a cell includes: a nanowire waveguide; an optical fiber connected to one end of the nanowire waveguide; and the genetic material with which the surface of the nanowire waveguide is coated.

### CROSS-REFERENCE TO RELATED APPLICATIONS

This application claims priority based on Korean Patent Application No. 10-2024-0027987 filed Feb. 27, 2024, the entire disclosure of which is incorporated herein by reference.

### INCORPORATION BY REFERENCE OF SEQUENCE LISTING

The content of the electronically submitted sequence listing, file name: Q302984_sequence listing as filed.XML; size: 7,130 bytes; and date of creation: Oct. 10, 2024, filed herewith, is incorporated herein by reference in its entirety.

### TECHNICAL FIELD

The present invention relates to a nanowire-based inoculation system for delivering genetic material into a single living cell and a method for fabricating the same. Specifically, the present invention relates to a nanowire-based inoculation system capable of delivering genetic material into a desired location in a single cell through evanescent field-driven release of the genetic material from the nanowire surface.

### BACKGROUND ART

In cell population and single-cell studies, intracellular drug delivery methods include viral and non-viral delivery methods. First, in the viral delivery method, a gene is inserted into a virus and then delivered into cells through the cell penetration mechanism of the virus. Due to this mechanism, the virus and the gene are hardly degraded and lost during the penetration process, and they can efficiently cross the cellular barrier, thereby exhibiting high transformation efficiency (Appraisal for the potential of viral and nonviral vectors in gene therapy: A review. Genes, 2022, Nrl knockdown by AAV-delivered CRISPR/Cas9 prevents retinal degeneration in mice. Nature Communications, 2017). However, the types and amounts of drugs that can be delivered are limited, and problems associated with intracellular immune response may occur. In addition, because the possibility of mutagenesis varies depending on the type of cell, control against mutagenesis is necessary (Mutation Research/Reviews in Genetic Toxicology, 1986). Above all, since the viral delivery method almost relies on passive gene delivery (e.g., diffusion) and spontaneous cellular uptake, which performs random material delivery, this method is not suitable for single-cell studies that require precision, and furthermore, the heterogeneity of cells having different characteristics cannot be considered, so the delivery efficiency is significantly non-uniform (Science, 2012, 336.6080:425-426; Annual Review of Cell and Developmental, 2009, 25.1:301-327; Nature reviews Molecular cell biology, 2011, 12.2:119-125).

Due to these limitations, despite the high efficiency of the viral delivery method, interest has been focused on the non-viral delivery method that has relatively low mutagenesis rates, as well as low cytotoxicity, and can expand the range of drug types. The non-viral delivery method can be broadly divided into a chemical method and a physical method, in which representative examples of the former include lipid- or polymer-based gene delivery. These are methods that utilize foreign substances or strong cationic substances that can interact with the cell membrane to deliver a desired drug into the cell. However, these substances cause cell immunity and cytotoxicity problems (Application of non-viral vectors in drug delivery and gene therapy. Polymers, 2021). In addition, the types of cells that can be transfected are also limited. In particular, similar to the problems of the viral delivery method, the non-viral delivery method relies on passive/spontaneous drug delivery, and thus performs random and non-uniform drug delivery, suggesting that there are still technical limitations in single-cell level studies.

To overcome the various problems mentioned above, electroporation and microinjection techniques, which apply physical shocks or changes to cells, have been developed. These techniques include a technique that increases the efficiency of material delivery by applying an electric field to cells to increase the permeability of the cell membrane, and a technique that delivers material while causing the most intuitive and physical contact by direct injection into cells. Through these techniques, it was possible not only to increase the drug delivery efficiency, but also to expand the range of possible transfection and overcome mutagenesis and cytotoxicity problems. However, in the case of electroporation, it is not a delivery method based on perfectly direct cell membrane penetration, and thus there is a problem in that the delivery efficiency is non-uniform and there is still a problem in that cell selectivity for material delivery into single cells is insufficient. To overcome these problems, techniques using acoustics (Scientific reports, 2017, 7.1:5275), optics (Journal of the Royal Society Interface, 2010, 7.47:863-871), and fluid dynamics (Scientific reports, 2016, 6.1:23937) have been developed. Although these techniques have ensured single-cell selectivity, they still have the disadvantage of being unable to ultimately avoid the non-uniformity of delivery efficiency because they deliver materials in a manner similar to electroporation.

Unlike these techniques, in the case of microinjection, it is a material delivery technique based on direct cell membrane penetration, and thus there is no need to consider problems caused by cell heterogeneity. In addition, since it is possible to select only the desired cells and deliver material into the cells without any restrictions on the type of substance, the microinjection technique is most commonly used in single-cell experiments among the many experiments mentioned above (Current opinion in biotechnology, 2008, 19.5:506-510.; Cloning & Stem Cells, 2001, 3.4:209-220; Human reproduction, 2014, 29.1:18-28). However, there is a limitation in that, since a micro-sized pipette is used, which is relatively large compared to the size of the cell, cell damage caused by direct cell membrane contact and penetration significantly reduces the cell viability (Physical non-viral gene 52-6 2024 Feb. 27 delivery methods for tissue engineering. Annals of biomedical engineering, 2013). In addition, the above technique has a disadvantage in that it cannot ignore external factors that can confuse experimental results, such as changes in the cell microenvironment due to pressure and fluid flow and induction of cell death, when material is to be delivered at a desired time (Yamauchi et al. 2007, Wall 2001). Accordingly, more precise next-generation drug delivery strategies are currently being designed by combining various experimental methods in order to overcome the problems of existing techniques (In vitro and ex vivo strategies for intracellular delivery, Nature, 2016). However, to date, a technique has not been developed which can ensure sufficient cell viability when used in single-cell experiments, and at the same time, can sufficiently perform stimulus response-based “on-demand” delivery that can provide a level of stimulation that is harmless and does not cause any collateral damage.

### DISCLOSURE

### Technical Problem

An object of the present invention is to provide a nanowire-based genetic material inoculation system capable of delivering various genetic materials into a single living cell. Specifically, an object of the present invention is to provide a nanowire-based genetic material inoculation system capable of efficiently manipulating the genetic characteristics of a single cell by directly inserting a genetic material-coated nanowire waveguide into the cell. Another object of the present invention is to provide a genetic material inoculation system which has a nanowire shape that may be easily inserted into a cell while minimizing damage to the cell membrane, and may rapidly deliver genetic material to a desired location in a cell within a short period of time (several seconds) using UV light, and a method for fabricating the same.

### Technical Solution

The above objects are achieved by a nanowire-based genetic material inoculation system for delivering a genetic material into a cell, including: a nanowire waveguide; an optical fiber connected to one end of the nanowire waveguide; and the genetic material with which the surface of the nanowire waveguide is coated.

Preferably, the surface of the nanowire waveguide may be coated with the genetic material by modifying the surface of the nanowire waveguide by a method selected from the group consisting of a biotin-avidin interaction, a copper-free click chemical reaction, NHS/EDC covalent bonding, and an aldehyde-amine reaction, and attaching the genetic material, linked to a photocleavable linker, to the modified surface of the nanowire waveguide.

Preferably, the nanowire waveguide may be fabricated by bringing a nanopipette, filled with a polyvinyl halide derivative solution containing poly(vinylbenzyl azide) (PVBN₃), polyvinyl halide, or a mixture thereof, into contact with the tip of the optical fiber, and then withdrawing the nanopipette.

Preferably, the photocleavable linker may be a nitrobenzyl-based photocleavable linker.

Preferably, when UV light is irradiated into the optical fiber, the UV light reaching the nanowire waveguide may form an evanescent field, so that the genetic material may be released into the cell.

Preferably, the UV light has a wavelength of 300 to 400 nm.

Preferably, the nanowire waveguide has a diameter of 100 nm to 500 nm.

Preferably, the genetic material includes a nucleic acid selected from the group consisting of mRNA, siRNA, miRNA, DNA, and a DNA-RNA duplex.

The above objects are also achieved by a method for fabricating a nanowire-based genetic material inoculation system, including steps of: fabricating a nanopipette; fabricating a tapered optical fiber by tapering one end of an optical fiber; filling the nanopipette with a polyvinyl halide derivative solution containing poly(vinylbenzyl azide) (PVBN₃), polyvinyl halide, or a mixture thereof, and bringing the nanopipette into contact with the tip of the optical fiber; fabricating a nanowire waveguide by evaporating the solvent from the polyvinyl halide derivative solution while withdrawing the nanopipette in a direction away from the optical fiber; and coating the surface of the nanowire waveguide with a genetic material by dipping the nanowire waveguide sequentially in a dibenzocyclooctyne-PEG-4-biotin solution and a streptavidin solution, followed by dipping in one selected from the group consisting of a solution containing a biotin-modified genetic material with a photocleavable linker based on biotin-avidin interaction, a solution containing the biotin-modified genetic material without the photocleavable linker, a solution containing a fluorescent substance and the biotin-modified genetic material with the photocleavable linker, and a solution containing the fluorescent substance and the biotin-modified genetic material without the photocleavable linker,

The above objects are also achieved by a nanowire-based genetic material inoculation method, including steps of: preparing the nanowire-based genetic material inoculation system; inserting the nanowire waveguide into a cell; and irradiating UV light through the optical fiber to release the genetic material into the cell.

Preferably, when UV light is irradiated into the optical fiber, the UV light reaching the nanowire waveguide may form an evanescent field, so that the genetic material may be separated from the surface of the nanowire waveguide and released into the cell.

Preferably, the cell may be a single living cell, and the nanowire waveguide may pass through the cell membrane, nuclear membrane, or organelle membrane within the cell without killing the cell, and release the genetic material at a desired location in the intracellular environment.

### Advantageous Effects

When the nanowire-based genetic material inoculation system according to the present invention is used, it is possible to inject a desired genetic material into a single living cell, thereby temporarily or permanently manipulating the genetic characteristics of the cell. Thus, it is possible to select desired cells and genetically manipulate and culture the cells. In addition, because selective analysis of individual cells is possible, it will become possible to study special and specific mechanisms of single cells that were difficult to identify in cell population studies.

### BRIEF DESCRIPTION OF DRAWINGS

FIGS. 1a and 1b schematically show the design of a nanowire-based genetic material inoculation system. Specifically, FIG. 1a shows an inoculation system for on-demand injection of genetic material into a single living cell. The 3-axis micromanipulator with 250 nm resolution allows precise positioning and spatiotemporal selective insertion of a nanowire into a cell cultured on a plate. After insertion of the nanowire, light from a UV light source is transmitted to the nanowire waveguide through an optical fiber without optical loss, and is transmitted to the surface of the nanowire based on an optical phenomenon, so that the genetic material is released. FIG. 1b shows nanowire penetration into the cell membrane and subsequent genetic material release induced by the evanescent field on the nanowire surface. As the nanowire is mechanically strong and thin enough, it can readily penetrate into the desired location in the cell membrane without cellular damage. The light transmitted through the optical fiber propagates through the nanowire waveguide, inducing light-triggered release of the gene from the nanowire surface.

FIGS. 2a-2d show a process of fabricating a nanowire on the tip of a tapered optical fiber. Specifically, FIG. 2a shows that a glass nanopipette filled with a solution is coaxially aligned with a tapered optical fiber. FIG. 2b shows that the nanopipette is pulled down to contact the tip of the optical fiber. FIG. 2c shows that the nanopipette is subsequently pulled up in a vertical direction, leading to growth of the PVBN₃ nanowire. FIG. 2d shows a freestanding nanowire fabricated on the tip of the tapered optical fiber. Scale bar: 10 μm.

FIG. 3 shows evaluation of the mechanical strength of a nanowire. The bright field image shows the process of inserting the nanowire into 1 wt % agarose gel. The maintenance of the nanowire shape after insertion demonstrates that the robustness of the PVBN₃ nanowire is enough to penetrate the cell membrane. Scale bar: 10 μm.

FIG. 4 shows schematic illustration of the on-demand nanowire surface modification process. First, a freestanding nanowire is fabricated by direct 3D writing using a glass nanopipette filled with a PVBN₃ solution. Then, the nanowire surface is modified with streptavidin by applying DBCO-azide click reaction and biotin-streptavidin interaction, consecutively. Finally, a biotin-modified gene containing a photocleavable linker is conjugated to the nanowire surface by biotin-streptavidin interaction. The genetic material on the nanowire surface can be readily released by UV exposure, allowing spatiotemporally controlled gene delivery into a single living cell.

FIG. 5 shows estimation of the concentration of the genetic material on the nanowire surface. For estimation, an aqueous solution (0.5 μL) of an oligonucleotide labeled with Cy3 fluorescent dye was deposited on a glass substrate. After solvent evaporation, a fluorescent image of the droplet was obtained (top left). Scale bar: 200 μm. In addition, a fluorescent image of the nanowire modified with the Cy3-labelled oligonucleotides was acquired (top right). Scale bar: 20 μm. In the images, the areas with fluorescent signal were automatically recognized by the binarization process (bottom). Based on the proportionality of total fluorescent intensity (average fluorescent intensity (I)×total area (A)) and the total number of fluorescent molecules (N), the concentration of the genetic material on the nanowire surface was calculated as approximately 0.017±0.001 molecules nm⁻².

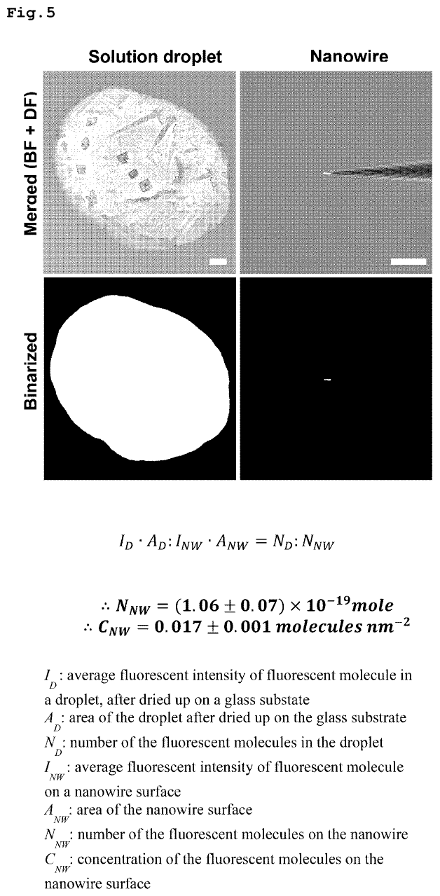

FIGS. 6a-6e show characterizations of a PVBN₃ nanowire. Specifically, FIG. 6a shows an FE-SEM image of a freestanding PVBN₃ nanowire grown on at the tip of a tapered optical fiber. Scale bar: 5 μm. FIG. 6b shows schematic illustration of light-responsive gene release from the nanowire surface. When UV light is transmitted to the nanowire, it propagates through total internal reflection at the interface. As the UV light is localized in the vicinity of the nanowire surface by forming an evanescent field, optical damage during nanowire insertion into living cells can be minimized. The genetic material on the nanowire surface can be released by the photocleavage reaction induced by the evanescent field. FIG. 6c shows the relative intensity of the evanescent field depending on the normal distance from the nanowire surface. The depth of the evanescent field (d_{50%}=49.2 nm) is large enough to induce photocleavage reaction on the nanowire surface. FIG. 6d shows the refractive index map of PVBN₃ nanowires in an aqueous solution for simulation of light propagation. Scale bar: 1 μm. FIG. 6e shows simulating light propagation through virtual conditions of the refractive index map of PVBN₃ nanowires in aqueous solution. Scale bar: 1 μm.

FIGS. 7a-7f show optical loss evaluation of a PVBN₃ nanowire waveguide. Specifically, FIG. 7a shows a transmission electron microscopy (TEM) image of the nanowire. The smooth surface of the PVBN₃ nanowire enables efficient light propagation with minimal optical loss. Scale bar: 500 nm. (Inset scale bar: 50 nm). FIG. 7b shows a bright field image of the nanowire that guides UV light transmitted from the tapered optical fiber. Scale bar: 10 μm. FIG. 7c shows a dark field image of the nanowire. Light scattering is negligible at the junction (yellow dashed circle), while it is observed only at the tip of the nanowire (white dashed circle) by sufficient light propagation in the nanowire without optical loss. Scale bar: 10 μm. FIG. 7d shows a schematic illustration and optical microscope image of optical power measurement at a tip of a bare optical fiber. Scale bar: 10 μm. FIG. 7e shows optical power measurement at a tip of the nanowire. Scale bar: 10 μm. FIG. 7f shows coupling efficiency depending on input laser power. (Coupling efficiency (red)=output laser power at the bare optical fiber tip (green)/output laser power at the nanowire tip (blue)=94.4±1.6%).

FIGS. 8a and 8b show evaluation of light-responsive gene release from the nanowire surface. Specifically, FIG. 8a shows bright field and fluorescence images of the nanowires modified with Cy5 DNA before (top) and after (bottom) UV exposure for 100 seconds. For the demonstration of light-responsive gene release from the nanowire surface, the oligonucleotides with (right) and without (left) a photocleavable (PC) linker were conjugated to the PVBN₃ nanowire surface and immersed in 1×PBS. Then, the fluorescent intensity of the surface of the nanowires was measured at 1-second intervals with UV light exposure. Scale bar: 20 μm. FIG. 8b shows normalized photoluminescence (PL) intensity with time during gene release induced by UV exposure from 10 seconds. The rates of gene release by photocleavage were different depending on the type and intensity of UV light source.

FIG. 9 shows spatioselective insertion of a PVBN₃ nanowire waveguide into a single living cell. (t₁-t₃) Nanowire insertion into the nucleus of a HeLa cell. (t₄-t₆) Nanowire insertion into the cytosol of the cell. The small diameter and high robustness of the nanowire allows nanowire penetration into a specific location in the cell membrane without cellular damage. Scale bar, 20 μm.

FIG. 10 shows identification of silenced cells after siRNA transfection into turbo GFP-expressing Hela cells. After fluorescence image acquisition (Step 1), all pixel intensities were collected and a threshold value was allocated to obtain binary images (Step 2). Then, the pixels corresponding to cell regions were automatically recognized by applying a binary image filter that removes salt and pepper noise (Step 3). Subsequently, histograms were extracted from the pixels in the cell regions (Step 4). The threshold intensity for distinguishing cells whose fluorescent intensity was reduced by GFP silencing was empirically determined from the histogram data analysis. Finally, the cell regions on the images were classified through multi-thresholding, and then normal cells and silenced cells were successfully segmented from the original cell images by using opening and closing algorithm (Step 5). Scale bar: 100 μm.

FIG. 11 shows investigation of GFP silencing 72 hours after DsiRNA transfection. GFP silencing g was investigated using fluorescence confocal images (top), automatically segmented images (middle), and their intensity histograms (bottom). As demonstrated by the fact that the area of magenta regions on the segmented images is large, both DsiRNA sequences (Seq No. 1 and Seq No. 2) induced GFP silencing in approximately 50% of turbo GFP-expressing Hela cells. Compared to the results of the control experiment, the peak of the intensity histogram shifted to the left after DsiRNA transfection due to the increase in the proportion of silenced cells with low PL intensity. Scale bar: 200 μm.

FIGS. 12a and 12b show investigation of GFP silencing over time after DsiRNA transfection. Specifically, FIG. 12a shows merged (bright: field+dark field) images of turbo GFP-expressing Hela cells over time after DsiRNA transfection (top) and their segmented images (bottom). FIG. 12b shows the ratio of silenced cell area to total cell area with time after DsiRNA transfection.

FIGS. 13a and 13b show the in situ process of local GFP silencing by inoculation of DsiRNA through nanowire insertion into an arbitrarily selected turbo GFP-expressing HeLa cell. Specifically, FIG. 13a shows nanowire surface modification with DsiRNA. FIG. 13b shows schematic illustration of nanowire-based inoculation of DsiRNA into a living cell for GFP inhibition. FIG. 13c shows observation of the genetic manipulation of cells by transfer of genetic material over time. As cells divide over time, the amount of green fluorescent protein in the parent cell and daughter cells decreases due to gene silencing, which reduces the green fluorescence in the cells. The red triangles represent the parent cells inoculated with the gene, and the cells marked with white solid lines are daughter cells. Scale bar: 20 μm.

FIG. 14 shows the mechanism of GFP silencing in a mammalian cell after inoculation of DsiRNA.

FIG. 15 shows green fluorescent protein (GFP) expression in Hela cells. The control (top) and GFP expression (bottom) images were obtained 24 hours after transfection with lipofectamine. Scale bar: 100 μm.

FIG. 16 shows staining-free cell viability measurement of Hela cells cultured at different pHs (pH 4.3, 5.0, and 6.0). Entropy-based image segmentation facilitates the distinction between live (green, bottom) and dead (red, bottom) cells on grayscale cell images (top) without an additional staining process. Due to the high local complexity of the dead cell area, larger entropy values are assigned to the pixels of the area. The ratio of dead cell area to total cell area on the segmented images was quantitatively measured. R_{pH4.3}=39.4%, R_{pH5.0}=7.5%, R_{pH6.0}=5.1%). Scale bar: 100 μm.

FIGS. 17a-17c show the in-situ process of local GFP expression by inoculation of plasmid DNA through nanowire insertion into an arbitrarily selected HeLa cell. Specifically, FIG. 17a shows nanowire surface modification with plasmid DNA. FIG. 17b shows schematic illustration of nanowire-based inoculation of plasmid DNA into a living cell for GFP expression. FIG. 17c shows observation of the genetic manipulation of cells by transfer of genetic material with time. As cells divide over time, the amount of green fluorescent protein within the cells increases due to gene expression, which increases the green fluorescence in the cell. Cells marked with the white solid line are daughter cells. Scale bar, 20 μm.

FIG. 18 shows the mechanism of GFP expression in a mammalian cell after inoculation of plasmid DNA.

### MODE FOR INVENTION

All technical terms used in the present invention, unless otherwise defined, are as defined below and have the same meaning as commonly understood by those skilled in the art to which the present invention pertains. In addition, although preferred methods or samples are described herein, those similar or equivalent thereto are also included in the scope of the present invention.

The present invention relates to a nanowire-based genetic material inoculation system for delivering genetic material into a cell and a method for fabricating the same.

Specifically, the nanowire-based genetic material inoculation system according to the present invention includes: a nanowire waveguide; an optical fiber connected to one end of the waveguide; and a genetic material with which the surface of the nanowire waveguide is coated. In particular, the present invention relates to a genetic material inoculation system that maximizes spatiotemporal selectivity while minimizing cell damage.

As used herein, the term “cell” includes all structural basic units existing in a living organism, such as mitochondria, chloroplasts, nuclei, endoplasmic reticulum, Golgi apparatus, lysosomes, secretory granules, secretory vesicles, phagosomes, and peroxisomes. In addition, the term “cell” as used herein includes not only living cells but also dead cells. In addition, the term “cell” as used herein may include not only single cells but also cell populations.

As used herein, the term “genetic material” refers to mRNA, tRNA, rRNA, DNA, DNA-RNA duplex, etc.

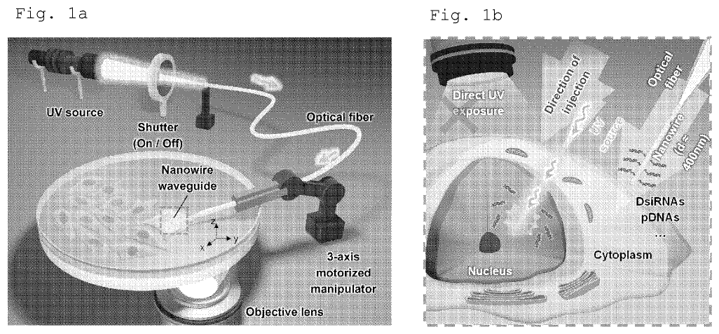

FIGS. 1a-1b schematically show a nanowire-based genetic material inoculation system according to the present invention. FIG. 1a shows an inoculation system for on-demand injection of genetic material into a single cell. As shown therein, the inoculation system includes: a nanowire waveguide; an optical fiber connected to one end of the nanowire waveguide; and a genetic material with which the surface of the nanowire waveguide is coated. Using a 3-axis micromanipulator with 250 nm resolution, the nanowire waveguide connected to one end of the optical fiber may be selectively inserted into a precise location of a cell cultured on a plate. After insertion of the nanowire waveguide, UV light emitted from the UV light source is transmitted to the nanowire waveguide through the optical fiber without optical loss, and is transmitted to the surface of the nanowire based on an optical phenomenon, so that the genetic material is released. As the nanowire waveguide according to the present invention is mechanically strong and thin enough, it can readily penetrate into the desired location in the cell without causing fatal damage to the cell, thus minimizing the size of membrane disruption. As the light is directed through the optical fiber coupled to the nanowire, the light exposure area of the cell may be minimized and near-field optical interactions may be induced locally at the nanowire surface. The UV light transmitted through the optical fiber propagates into the nanowire waveguide and reacts with a photocleavable (PC) linker present on the nanowire surface by an evanescent t field, inducing the release of the genetic material. This genetic material inoculation system may immediately release the genetic material into a living cell at a desired time through in-situ nanowire insertion and local UV exposure without cellular damage.

The nanowire waveguide according to the present invention refers to a nano-sized conducting wire for detecting light (optical signal), and may have a diameter from less than 100 nm to several hundred nm. For example, the diameter (d) of the nanowire waveguide may be 300 nm to 500 nm, preferably 350 nm to 450 nm. In particular, if the diameter is less than 300 nm, the amount of the fixed genetic material may be limited, and if the diameter is more than 900 nm, the nanowire waveguide may cause cellular damage. The diameter of the nanowire waveguide may vary depending on the wavelength of the light to be guided, the material to be delivered, etc.

The nanowire waveguide for intracellular delivery of genetic material may be fabricated by direct 3D fabrication of a polymer precursor and subsequent modification of the nanowire surface. In the 3D fabrication process, the precursor is deposited at the tip of a tapered optical fiber by solvent evaporation to form a polymer nanowire optically assembled with the optical fiber. For precise fabrication of the nanowire, the growth of the nanowire is monitored in real time using a side-view optical microscope system.

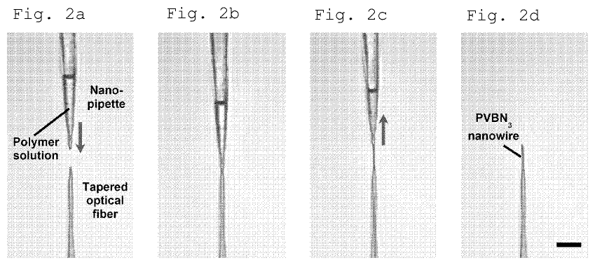

FIGS. 2a-2d show a process for fabricating a nanowire waveguide according to the present invention, which is monitored in real time using an optical microscope system. As shown in FIGS. 2a-2d, in the 3D fabrication process, a polymer precursor is deposited at the tip of a tapered optical fiber by solvent evaporation to form a polymer nanowire optically assembled with the optical fiber. Specifically, a nanopipette is fabricated, and a tapered optical fiber is fabricated by tapering one end of an optical fiber. Then, the nanopipette is filled with a polymer precursor solution, preferably a poly(vinylbenzyl azide) solution, and brought into contact with the tip of the optical fiber. Next, the solvent of the polymer solution is evaporated while the nanopipette is withdrawn in a direction away from the optical fiber, thereby fabricating a nanowire waveguide.

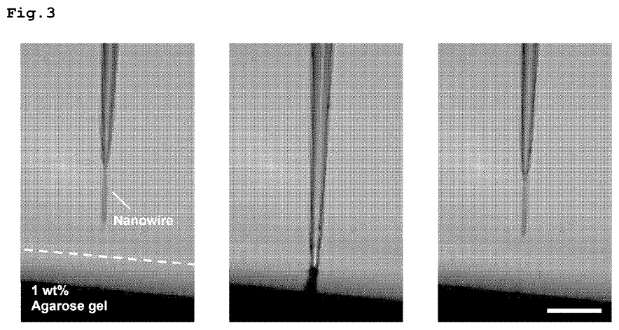

The polymer precursor used in the fabrication of the nanowire waveguide may be a polyvinyl halide derivative solution containing poly(vinylbenzyl azide) (PVBN3), polyvinyl halide, or a mixture thereof. Preferably, the polymer precursor may be poly(vinylbenzyl azide) (PVBN₃). Poly(vinylbenzyl azide) (PVBN3) has three unique properties: mechanical robustness, high refractive index, and chemical programmability. First, the excellent Young's modulus (E≈1.7 Gpa) of PVBN₃ (M.-J. Yong, B. Kang, U. Yang, S. S. Oh, J. H. Je, Live streaming of a single cell's life over a local pH-monitoring nanowire waveguide. Nano Lett. 2022, 22, 6375) facilitates the insertion of nanowires into cells, as demonstrated by their deformation-free penetration into a rigid gel matrix with a strength similar to that of the cell membrane (FIG. 3) (M. Ahearne, Y. Yang, A. J. El Haj, K. Y. Then, K.-K. Liu, Characterizing the viscoelastic properties of thin hydrogel-based constructs for tissue engineering applications. J. R. Soc. Interface 2005, 2, 455). Second, since the refractive index of PVBN₃ (n≈1.71) (C.-M. Tsai, S.-H. Hsu, C.-C. Ho, Y.-C. Tu, H.-C. Tsai, C.-A. Wang, W.-F. Su, High refractive index transparent nanocomposites prepared by in situ polymerization. J. Mater. Chem. C 2014, 2, 2251) is higher than that in the cellular environment (n≈1.37), UV light may be completely reflected at the interface while propagating within the nanowire, forming an evanescent field, which enables local light modulation.

Finally, the azide moiety of PVBN₃ facilitates the modification of the nanowire waveguide surface with the genetic material.

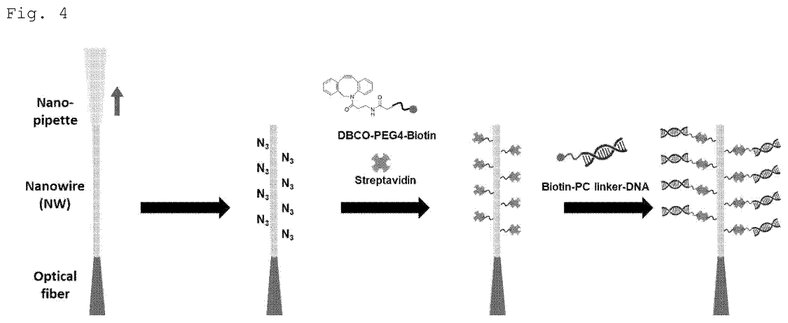

Nanowire waveguide surface modification was performed by dipping the nanowire sequentially in solutions containing DBCO-PEG4-biotin, streptavidin, and predesigned genetic material, respectively (FIG. 4). The DBCO-azide click reaction (K. Kettenbach, T. L. Ross, A 18 F-labeled dibenzocyclooctyne (DBCO) derivative for copper-free click labeling of biomolecules. Med. Chem. Commun. 2016, 7, 654) and the biotin-streptavidin reaction (P. C. Weber, D. H. Ohlendorf, J. J. Wendoloski, F. R. Salemme, Structural origins of high-affinity biotin binding to streptavidin. Science 1989, 243, 85) provide a robust connection between the nanowire surface and the genetic material to be delivered, allowing for high surface concentrations of the genetic material (0.017±0.001 molecules nm⁻²).

According to one embodiment of the present invention, the surface of the nanowire waveguide may be coated with the genetic material by modifying the surface of the nanowire waveguide by a method such as biotin-avidin interaction, copper-free click chemical reaction, NHS/EDC covalent bonding, aldehyde-amine reaction, etc., and attaching the genetic material, linked to a photocleavable linker, to the modified surface of the nanowire waveguide.

According to one embodiment of the present invention, the surface of the nanowire waveguide may be coated with the genetic material by dipping the nanowire waveguide sequentially in a dibenzocyclooctyne-PEG-4-biotin solution and a streptavidin solution, followed by dipping in one selected from the group consisting of a solution containing a biotin-modified genetic material with a photocleavable linker based on biotin-avidin interaction, a solution containing the biotin-modified genetic material without the photocleavable linker, a solution containing a fluorescent substance and the biotin-modified genetic material with the photocleavable linker, and a solution containing the fluorescent substance and the biotin-modified genetic material without the photocleavable linker.

Preferably, to form a binding site, to which a genetic material can bind, on the surface of the nanowire waveguide, DBCO-biotin is bound to the surface through azide-DBCO click reaction, and then streptavidin is bound to the surface. Thereafter, a nucleic acid that serves to capture the genetic material is chemically bound to photocleavable (PC) biotin, a photocleavable linker that responds to UV light. Finally, DsiRNAs or plasmids are systematically linked using complementary linkage according to the nucleotide sequence.

The photocleavable linker may be a nitrobenzyl-based photocleavable linker (H. Zhao, E. S. Sterner, E. B. Coughlin, P. Theato, o-Nitrobenzyl alcohol derivatives: Opportunities in polymer and materials science. Macromolecules 2012, 45, 1723). In particular, as a nitrobenzyl-based photocleavable linker in place of azobenzene or spiropyran, which changes its molecular structure by photoisomerization, is bound to the genetic material, the genetic material may be released from the nanowire surface immediately after UV exposure.

According to the present invention, a strategy capable of stably delivering various substances has been established through nucleotide sequence-based binding that is freely designed. When UV light is irradiated through the optical fiber to the nanowire waveguide fabricated as described above, the UV light is totally reflected inside the waveguide and an evanescent field is formed only in a local area (tens of nm) on the surface. Thus, the UV exposure area in the cell may be minimized. In addition, the evanescent field can cleave the photocleavable linker on the nanowire waveguide surface, enabling immediate delivery of genetic material at a desired time. Based on this fact, in addition to overcoming the problem of fatal damage and death of living cells, the present inventors selected a single cell and performed immediate delivery of genetic material into the cell. This makes it possible to manipulate the genetic characteristics of a single living cell to be ultimately controlled.

The present invention will be described below in detail by way of examples, but the scope of the present invention is not limited by these examples.

### EXAMPLES

### Materials

Poly(vinylbenzyl chloride) (PVBC; a 60:40 mixture of 3- and 4-isomers; Mₙ: about 55,000), sodium azide, sodium chloride, magnesium chloride, a dibenzocyclooctyne-PEG-4-biotin conjugate (DBCO-biotin), deionized water, methanol, N, N-dimethylformamide (DMF), 1-methyl-2-pyrrolidinone (NMP), 10×phosphate-buffered saline (PBS), agarose power, propidium iodide, and 100×antibiotic/antimycotic were purchased from Sigma-Aldrich (St. Louis, MO). FBS was purchased form Gibco (Waltham, MA). Streptavidin was purchased from Promega (Madison, WI). Dulbecco's modified Eagle's medium (DMEM), penicillin and streptomycin were purchased from Welgene (Korea). Invitrogen lipofectamine 3000 transfection reagent was purchased from Lipidomia (Korea). DNA, RNA, and modified oligonucleotides were synthesized by IDT (Coralville, IA) (Table 1).

### Fabrication of Nanowire-Based Genetic Material Inoculator

30 mg PVBC was dissolved in anhydrous DMF solvent (0.6 mL) and sonicated for 10 min. Subsequently, 40 mg sodium azide was added to the PVBC solution to induce S_{N}2 reaction. As the azide ion is an excellent nucleophile, it can readily participate in the reaction, substituting the alkyl chloride in PVBC. After fluxing the mixed solution for 3 hours at 80° C., poly(vinylbenzyl azide) (PVBN₃) produced through the reaction was precipitated by adding 0.6 mL methanol. The precipitated PVBN₃ was separated by centrifuging at 13,500 rpm for 1 min (Mini-microcentrifuge, Labogene), and then remaining solution with excess unreacted sodium azide was removed. Thereafter, the precipitates were freeze-dried for 1 hour and then dissolved in 100 μL NMP solvent.

To fabricate a nanowire waveguide for the genetic material inoculator, a nanopipette and a tapered optical fiber were obtained by tapering a glass capillary tube and an optical fiber, respectively, by a programmed heating pulling process using a laser-based micropipette puller (P-2000, Sutter Instrument). Subsequently, the nanopipette and the tapered optical fiber were accurately positioned with a high spatial resolution of 250 nm by using a home-built 3D writing machine consisting of three-axis stepping motor stages (XA07A and ZA07A, Kohzu Precision). The nanopipette filled with PVBN₃ solution was co-axially aligned with the tapered optical fiber, and a nanowire waveguide was fabricated on the fiber tip by a direct 3D writing process. The fabrication of the PVBN₃ nanowire was controlled in real-time under a custom-made software while monitoring the whole fabrication process using a two-axis side-view optical microscope which is composed of an objective lens (100× Plan Apo Infinity Corrected Objective, Mitutoyo), a charge coupled device (CCD) camera (INFINITY 1-2C, Lumenera Camera), and a 590 nm LED light source (Precision LED spotlight, Mightex).

The surface of the nanowire waveguide was modified with the genetic material to be injected into cells. The modification of the nanowire surface was performed by a three-step process. First, the nanowire surface was biotin-functionalized by the DBCO-azide click reaction by dipping the nanowire in 1 μM DBCO-biotin solution. Then, the nanowire was dipped in a streptavidin solution (0.1 mg/mL in 1×PBS). Finally, a biotin-modified genetic material containing a photocleavable linker was conjugated on the nanowire surface by biotin-streptavidin interaction. Each dipping process for nanowire surface modification was performed for 1 min.

### Characterization of Nanowires

The nanoscale morphology of fabricated nanowires was analyzed using field emission scanning electron microscopy (FE-SEM, JSM-7800F Prime, JEOL). For the demonstration of a very smooth nanowire surface, PVBN₃ nanowires were directly fabricated in the lateral direction on a Lacey carbon film supported copper TEM grid. Then, the high-resolution images of the nanowire surface were obtained.

### Cell Experiment

HeLa cells (Korean Cell Line Bank) and turbo GFP-expressing HeLa cells (Spain, Innoprot) were used for cell experiments. Cells were cultured with DMEM supplemented with 10% FBS, 100 U/mL penicillin, and 100 μg/ml streptomycin, 100× antibiotic/antimycotic diluted at 1× in culture medium. The cells were incubated at 37° C. in 5% CO₂/95% air. Gene transfection or nanowire-based gene injection was carried out after culturing the cell lines for 2 to 3 days.

### Simulation of Light Propagation in Nanowire Waveguide

The numerical calculation of light propagation in the nanowire was performed using finite-difference time-domain (FDTD) simulation implemented by MATLAB software. Specifically, the simulation was performed for a PVBN₃ nanowire with a specific size (1=10 μm, d=400 nm), in which light with a wavelength of 360 nm was transferred to the nanowire through the optical fiber in an aqueous solution. The refractive index of all media was set as n_{nanowire}=1.71, nₛₒₗᵤₜᵢₒₙ=1.37, and n_{optical fiber}=1.5, respectively.

### Lipofectamine Assay

To measure the efficiencies of DsiRNA and plasmid DNA, HeLa cells and turbo GFP-expressing Hela cells were preincubated in 24-well plates with DMEM supplemented with 10% FBS at 37° C. in a humidified atmosphere with 5% CO₂ for 12 h to adapt cell culture environments before lipofectamine assay. Transfection with lipofectamine was carried out according to optimized instructions following incubating for 12 h. 25 μL DMEM without fetal bovine serum were mixed with 3 μL lipofectamine (Solution 1). 10 pmol of each of DsiRNA sense sequences (#1 and #2), DsiRNA antisense sequences (#1 and #2), GFP pDNA, and RFP pDNA was given to 25 μL DMEM without fetal bovine serum (Solution 2). The two solutions were mixed together using a digital orbital shaker (WiseMix, DAIHAN Scientific) for 20 min at room temperature. 30 μL of the mixture was then given to the cell cultures (final concentration of oligo was 10 nM) and incubated at 37° C. in a humidified atmosphere with 5% CO₂ for 6 h. After incubation, the cell cultures were washed with 1×PBS three times and then incubated with a culture medium containing 10% FBS and antibiotics.

### Staining-Free Cell Viability Analysis

To evaluate the effect of the pH of cell culture media on the cell viability, HeLa cells were cultured at 25° C. for 1 hour at controlled pHs of 4.3, 5.0, and 6.0, respectively. Then, the cells were washed with 1×PBS solution and the medium was replaced with fresh buffer. After incubation at 37° C., the bright field images of the cells were obtained using a confocal microscope (STELLARIS 5, Leica). From the obtained images, dead cells and live cells were automatically identified by MATLAB-based image processing. Specifically, the complexity of adjacent pixels was measured by applying entropy filtering to the gray-scale cell images. The entropy (E) in the pixels in bit units is defined by Formula 1 below.

$$E = - {\sum }_{x ∈ 𝒳} p ( x ) log p ( x ) \qquad [ Formula 1 ]$$

wherein p is the frequency of pixels with corresponding intensity range. Due to the high complexity of dead cell area, larger entropy values are assigned to the pixels of the area. By determining the threshold of the entropy value that distinguishes live and dead cells, live and dead cell areas were segmented. After applying noise fileting and smoothing to the segmented images using the opening and closing algorithm, the ratio of dead/live cell area was automatically measured by counting the pixels of the corresponding area.

### Gene Inoculation

The spatial position of the nanowire-based inoculator was precisely controlled by a three-axis micromanipulator consisting of stepping motor stages (XA07A and ZA07A, Kohzu Precision). Nanowire injection into a living cell was monitored in real-time using a confocal microscope (STELLARIS 5, Leica). When the nanowire was injected into the desire site in the cell, 360 nm UV light from the laser source (UV-FN-360-50 mW, CNI) was transmitted to the nanowire through the optical fiber, leading to light-induced gene release from the nanowire surface. After 5 seconds of exposure, the UV laser supply was cut off by closing the shutter, and the nanowire was removed from the cell. To analyze gene silencing or expression, the cell images were obtained every 24 hours after the inoculation.

### Results

### 1. Characterization of Evanescent-Field-Driven Gene Release

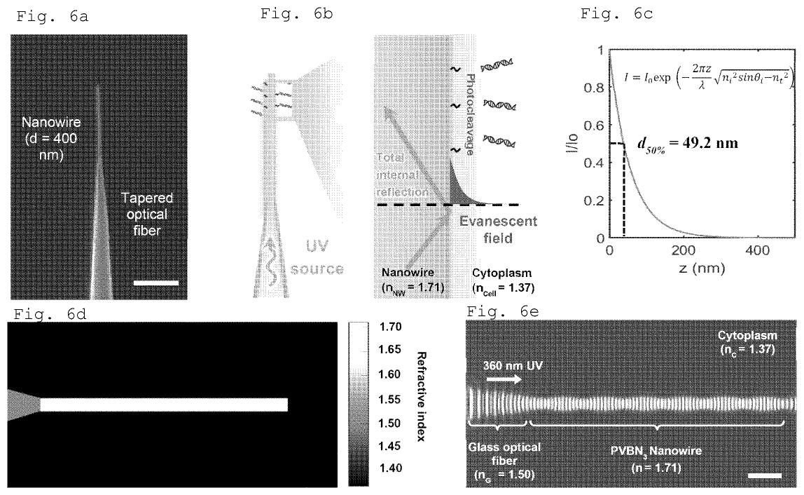

To investigate the morphological and optical characteristics of the PVBN₃ nanowire, microscopic imaging and computational simulation were conducted, respectively (FIGS. 6a-6e). The high-resolution image of a freestanding PVBN₃ nanowire grown on a tapered optical fiber demonstrates its high aspect ratio and thin diameter (d=400 nm), required for superior spatioselectivity (FIG. 6a). When UV light is transmitted to the nanowire, it is confined in the nanowire due to the total internal reflection, leading to formation of evanescent field on the surface (FIG. 6b). The evanescent field can lead to photocleavage reactions localized in a region of several tens of nanometers from the nanowire surface, and decreases exponentially as the distance from the surface increases (FIG. 6c). The result of finite-difference time-domain simulation of light propagation in the nanowire displays the confined propagation of UV light in the PVBN₃ nanowire (FIGS. 6d and 6e).

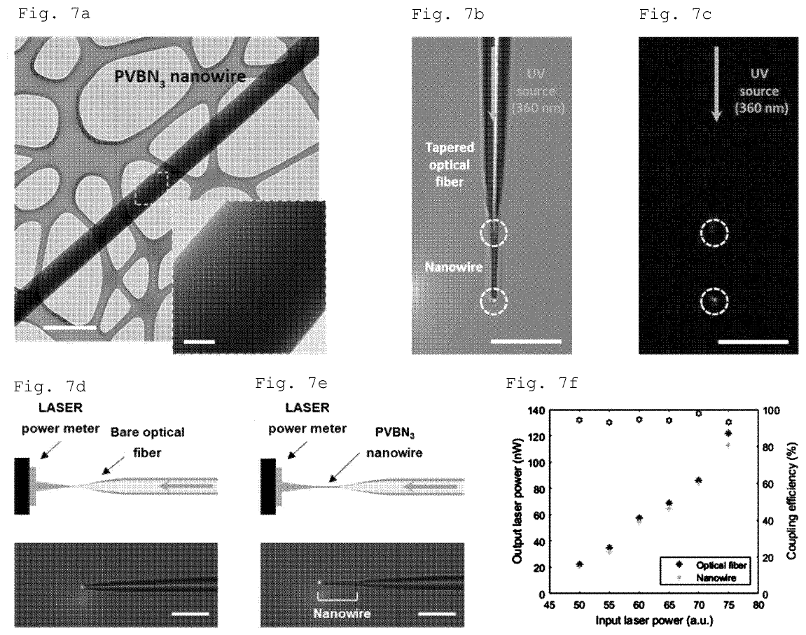

The intensity of the evanescent wave is sufficient to cleave the nitrobenzyl linker on the nanowire surface because of the efficient propagation of the light from the optical fiber to the nanowire without optical loss. In addition, the very smooth surface of the nanowire minimizes the optical loss during light propagation due to the minimized surface scattering (FIG. 7a). Due to the rigid structural assembly between the polymeric nanowire and the optical fiber, light scattering at the junction was rarely observed (FIGS. 7b and 7c). When compared with bare optical fiber (FIG. 7d), the decrease in the optical power measured at the tip of the nanowire combined with the tapered optical fiber (FIG. 7e) was negligible, indicating that the nanowire has high optical coupling efficiency (>94%) (FIG. 7f).

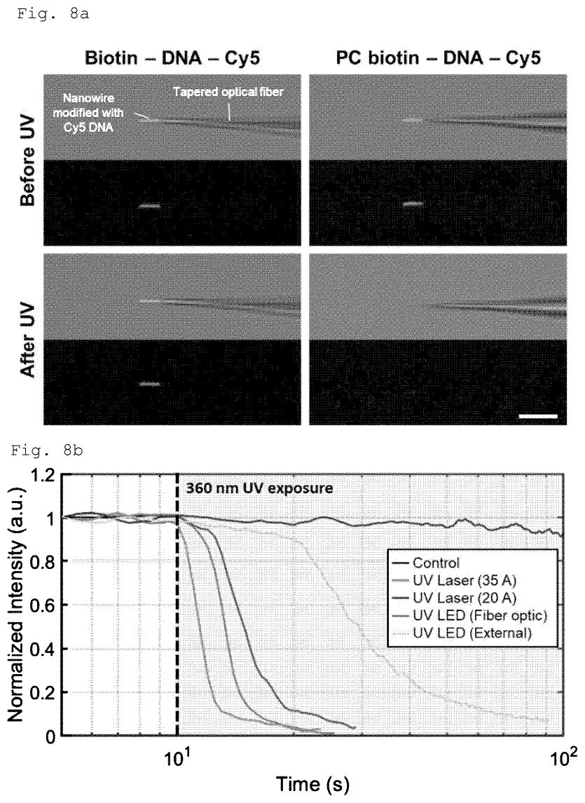

Despite the confined optical field with a nanoscale size, the genetic material on the nanowire surface can be released within a few seconds due to excellent optical coupling and efficient light propagation. To demonstrate the light-driven release of the genetic material, a set of nanowire waveguides were modified with pre-designed genetic materials (biotin-DNA-Cy5 and PC biotin-DNA-Cy5, respectively), and irradiated with UV light for 100 seconds while being immersed in 1×PBS solution (FIG. 8a). Then, the decrease in the photoluminescence (PL) signal was observed only on the nanowire modified with PC biotin-DNA-Cy5, indicating the release of the genetic material by photocleavage reaction. The normalized PL intensity with time was also investigated to evaluate the release rate of the genetic material (FIG. 8b). When light was irradiated through the optical fiber, the release rate of the genetic material varied depending on the type of light source. Interestingly, when a 35A UV laser source was used, more than 90% of the genetic material was released within 3.0 seconds, and the rate was almost 20 times faster than that in a conventional method of irradiating UV light from an external light source over a large area.

### 2. Single-Cell Gene Modification by Light-Responsive Gene Inoculation

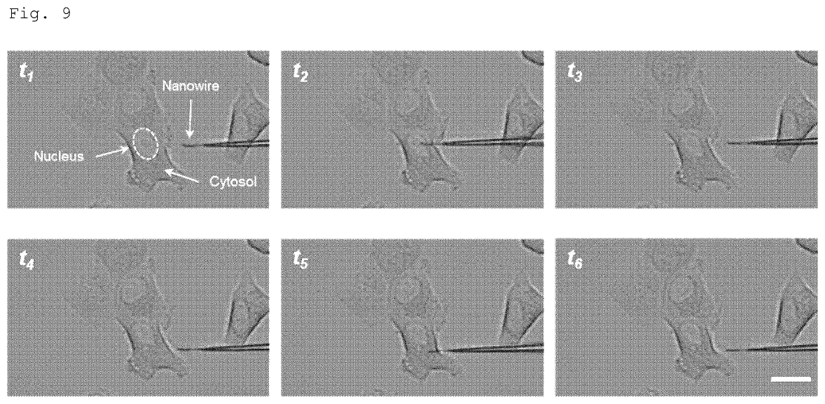

Genetic materials, such as DNA, mRNA, and siRNA, have their own roles in a cell, and many proteins that assist these roles are distributed in the regions partitioned by intracellular membranes. Therefore, an exogenous genetic material needs to be delivered to a desired site in a cell in order to fully exhibit its genetic function. For example, in order for synthetic DNA to be transcribed within a cell, the DNA needs to be injected into the nucleus, while mRNA needs to be located in the cytoplasm in order to perform protein translation functions (F. Crick, Central dogma of molecular biology. Nature 1970, 227, 561). In the case of the gene inoculation system according to the present invention, the nanowire waveguide can be freely inserted into the desired location in a single living cell without damage to the cell membrane due to its high aspect ratio and excellent mechanical strength, enabling the spatioselective delivery of genetic material (FIG. 9).

Dicer-substrate small interfering RNAs (DsiRNAs), which are artificial molecules for silencing genes of interest, possess up to 100-fold higher silencing efficiency than traditional siRNAs (S. Ramachandran, P. H. Karp, P. Jiang, L. S. Ostedgaard, A. E. Walz, J. T. Fisher, S. Keshavjee, K. A. Lennox, A. M. Jacobi, S. D. Rose, M. A. Behlke, M. J. Welsh, Y. Xing, P. B. McCray Jr., A microRNA network regulates expression and biosynthesis of wild-type and _F508 mutant cystic fibrosis transmembrane conductance regulator. Proc. Natl. Acad. Sci. 2012, 109, 13362; C. Dutta, N. Avitahl-Curtis, N. Pursell, M. Larsson Cohen, B. Holmes, R. Diwanji, W. Zhou, L. Apponi, M. Koser, B. Ying, D. Chen, λ. Shui, U. Saxena, W. A. Cyr, A. Shah, N. Nazef, W. Wang, M. Abrams, H. Dudek, E. Salido, B. D. Brown, C. Lai, Inhibition of glycolate oxidase with dicer-substrate siRNA reduces calcium oxalate deposition in a mouse model of primary hyperoxaluria type 1. Mol. Ther. 2016, 24, 770).

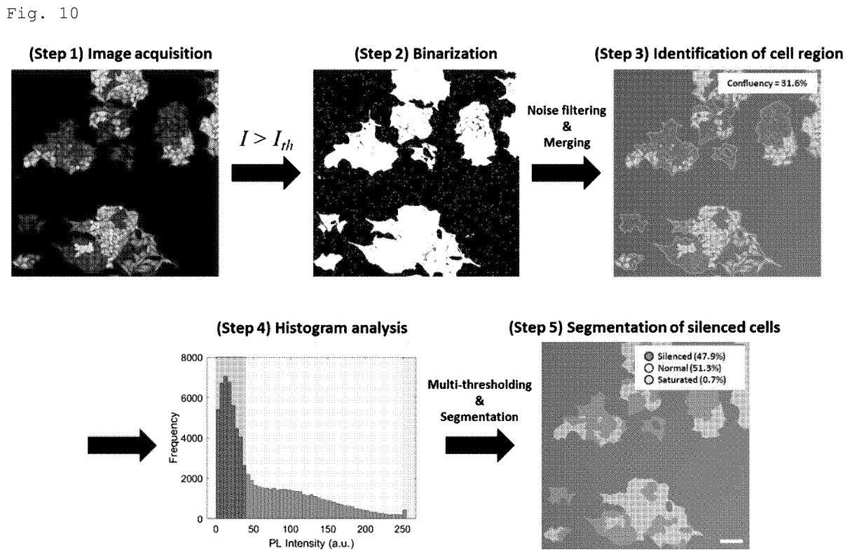

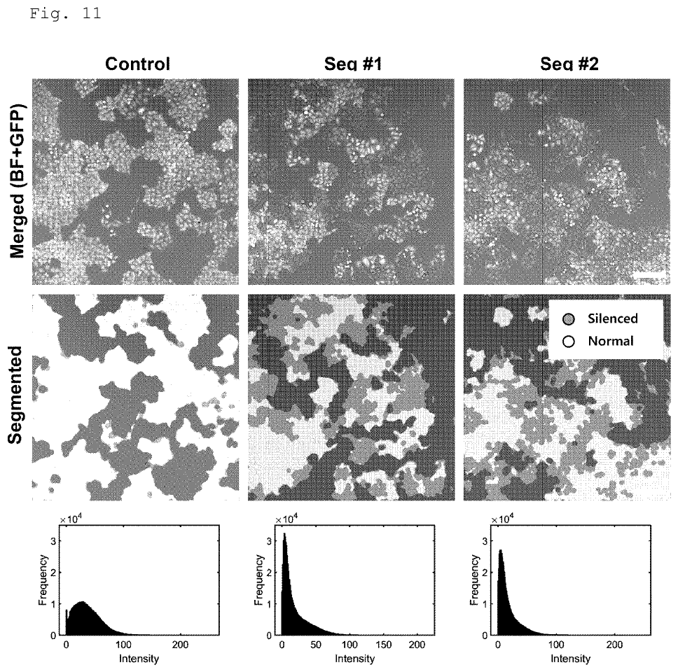

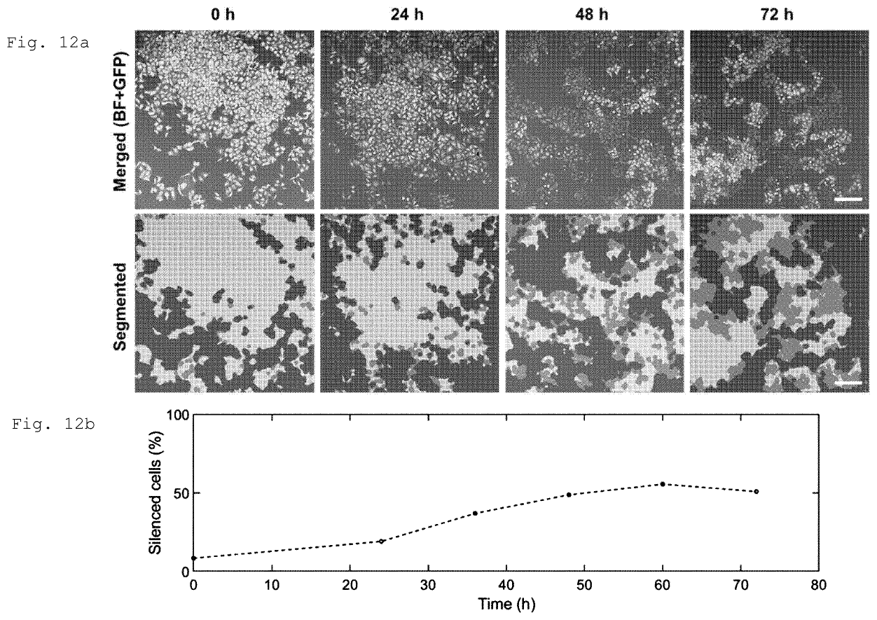

As an effective tool for sequence-specific gene silencing, DsiRNA was adopted to inhibit the expression of turbo GFP in a single living cell. Investigation of the GFP silencing ability of DsiRNA was conducted by lipofectamine-mediated transfection. After transfection of DsiRNA into turbo GFP-expressing HeLa cells, identification of silenced cells was automatically conducted via the thresholding-based image segmentation algorithm created using MATLAB (FIG. 10). To silence the expression of turbo GFP, two DsiRNAs targeting different sequences was designed (Seq #1 and Seq #2), and their silencing efficiency was observed 72 hours after transfection (FIG. 11). As demonstrated by the large area of magenta regions on the segmented images, both DsiRNA sequences induced GFP silencing in approximately 50% of turbo GFP-expressing Hela cells. Compared to the results of the control experiment, the peak of the intensity histogram shifted to the left after DsiRNA transfection due to the increase in the proportion of silenced cells with low PL intensity. The proportion of silenced cells was also investigated over time after DsiRNA transfection (FIGS. 12a-12b). The proportion of silenced cells increased up to about 60 hours after transfection and then gradually decreased, which is consistent with the timescale of siRNA-induced transient silencing known in previous studies.

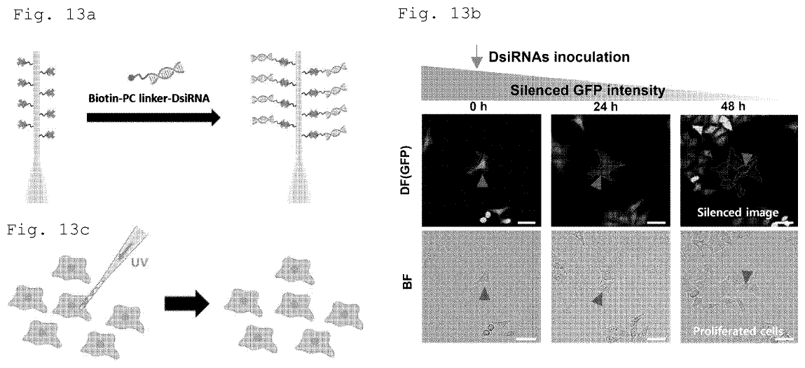

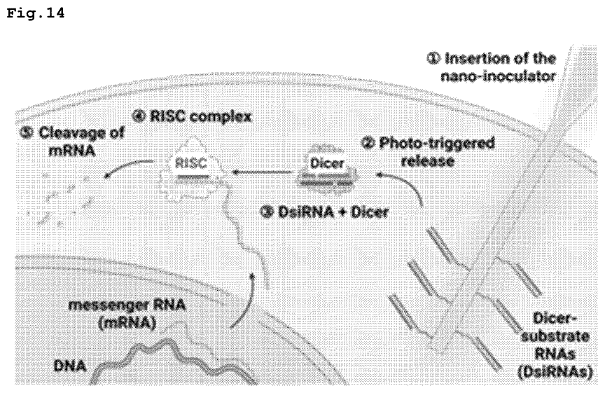

Unlike lipofectamine-mediated transfection which delivers genetic materials only to the arbitrary parts of cell populations, nanowire-based inoculation allows genetic modification of a selected living cell. In situ, nanowire-based inoculation of DsiRNA successfully induced GFP silencing of a user-selected cell (FIGS. 13a-13b). After insertion of the nanowire modified with DsiRNA (FIG. 13a), the genetic material on the nanowire surface was delivered into a GFP-expressing HeLa cell (FIG. 13b). After inoculating DsiRNA into the cytoplasm, the green fluorescence signal of the cells was measured, and after 48 hours, it was clearly reduced by inhibition of GFP expression only in the daughter cells of the cell injected with DsiRNA (FIG. 13c, yellow circles). The DsiRNA injected through the nanowire is cleaved into small fragments by the dicer in the cell, and then forms an RNA-inducing silencing complex (RISC), in which the target mRNA complementarily binds to siRNA and is then degraded, reducing GFP expression (FIG. 14). Indeed, the nanowire-based inoculation system allows the RISC metabolism to be induced only in a selected living cell.

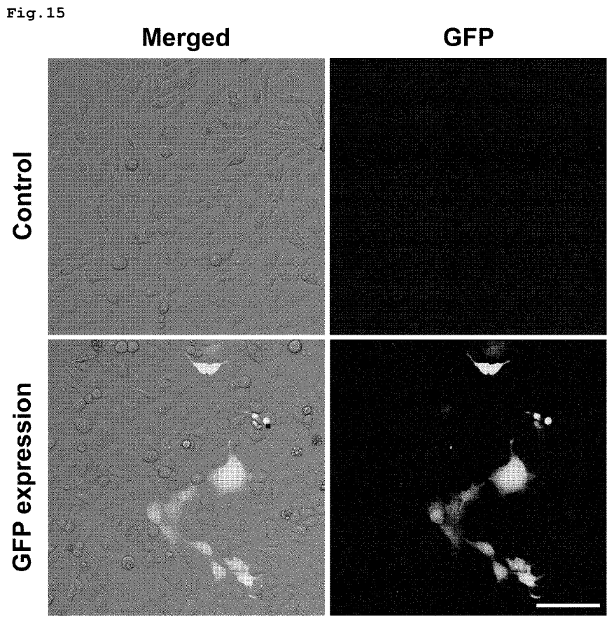

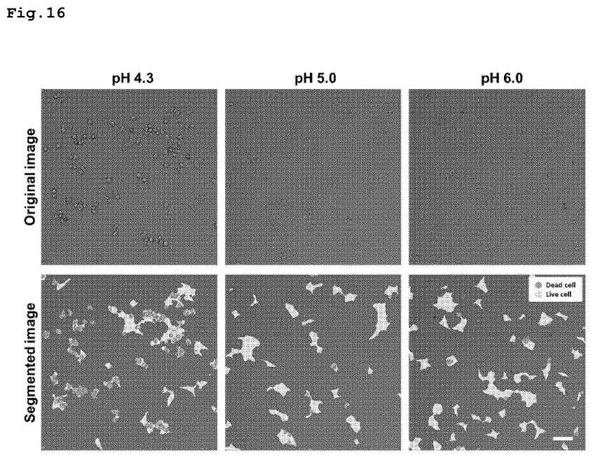

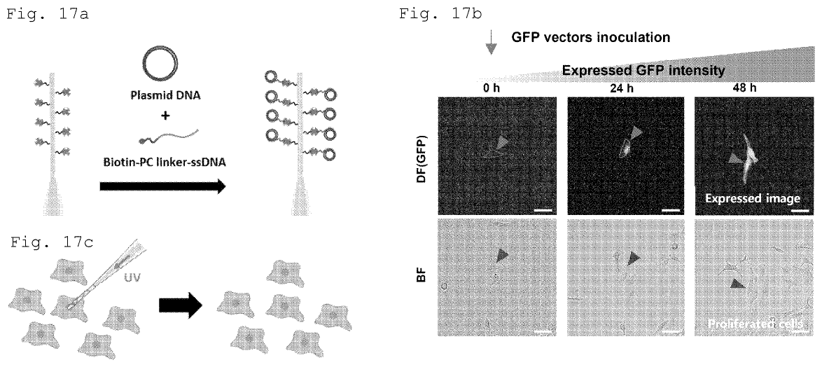

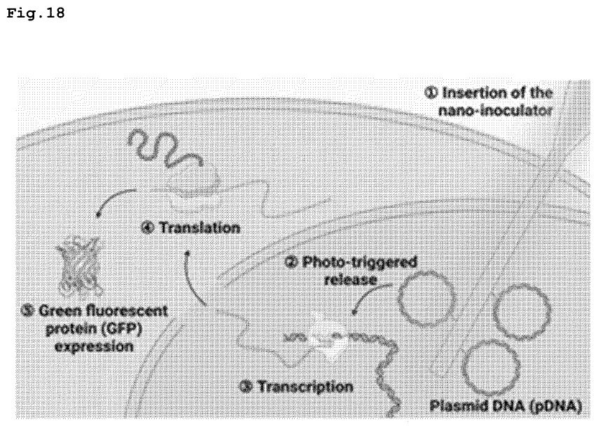

Inoculation of oligonucleotides can be extended to the delivery of diverse materials, including plasmid DNAs (pDNAs) or proteins, by designing target-specific DNA sequences such as aptamers. For example, a triplex-forming oligonucleotide (TFO) can be adopted to inject pDNA with a double helical structure into a living cell. As a material to be inoculated into a cell, pDNA capable of inducing GFP expression in a cell was employed (FIG. 15). In order to form a stable triplex structure on the surface of the nanowire, the TFO sequence was designed in consideration of various factors, such as pH, mismatch, and ion concentration, and the cell viability in the triplex forming milieu was also be investigated under the conditions described below (Table 1 and FIG. 16). With the nano-inoculation system combined with the triplex formation strategy, the plasmid DNA was successfully injected into the nucleus of a living HeLa cell, which led to GFP expression within the cell over time (FIGS. 17 and 18). In this way, it is expected that more diverse biological entities could be inoculated into a single living cell via the thorough design of genetic materials.

TABLE 1

| Name | Sequence (5′ to 3′) | Length (nts) |
|---|---|---|
| PC biotin-DNA- | [PC biotin] TTT CAC CCT TCA CTA GGT [Cy3] | 18 |
| Cy3 (Seq. No. 1) | | |
| PC biotin-DNA- | [PC biotin] TTT CAC CCT TCA CTA GGT [Cy5] | 18 |
| Cy5 (Seq. No. 2) | | |
| PC biotin- | [PC biotin] UUU UUG CGU GAU CUU CAC CGA CAA | 30 |
| DsiRNA sense | GAU CdAdT | |
| seq. 1 (Seq. No. 3) | | |
| PC biotin- | [PC biotin] UUU UUC AAC AAG AUG AAG AGC ACC | 30 |
| DsiRNA sense | AAA GdGdC | |
| seq. 2 (Seq. No. 4) | | |
| DsiRNA | AUG AUC UUG UCG GUG AAG AUC ACG CUG | 27 |
| antisense seq.1 | | |
| (Seq. No. 5) | | |
| DsiRNA | GCC UUU GGU GCU CUU CAU CUU GUU GGU | 27 |
| antisense seq. 2 | | |
| (Seq. No. 6) | | |
| PC biotin-TFO | [PC biotin] TTT TTT TTT TCT CCT TTC TCT TTC | |
| (Seq. No. 7) | TTC CCT TCC TTT CTC | 39 |

The present invention relates to a novel genetic material inoculation device capable of delivering genetic material into a single living cell without cellular damage, based on thorough control of the spatial location of a polymer nanowire, the binding method based on the nucleic acid nucleotide sequence, and the surface material. Since the nanowire waveguide according to the present invention is directly fabricated using the 3D fabrication method, the length thereof can be easily controlled, allowing quantitative control of the gene to be injected. In addition, when a very thin (<400 nm) and strong (approximately 1.7 GPa) nanowire is mechanically inserted, intracellular delivery of genetic material is possible regardless of the cell type. Although the nanowire-based inoculation system that targets only one cell at a time has the limitation of low throughput compared to other previous studies such as nanoneedle arrays or endocytosis, it has an excellent advantage in terms of high spatial selectivity. This inoculation system enables different biomaterials to be injected into the same cell through multiple inoculations, and is expected to help understand the previously unknown metabolisms of cellular organelles.

External manipulation of the nanowire surface material via evanescent waves is advantageous in terms of inducing spatially localized responses at the nanoscale and minimizing light-induced damage to cells. In the present system, light escapes from the nanowire tip, which can be resolved by simple capping with a material with high absorbance. Only in a localized region of tens of nanometers from the surface, the evanescent field can induce various optical interactions, such as optical coupling, optical trapping, photocleavage, and fluorescence resonance energy transfer (FRET). Since UV light is totally reflected inside the nanowire, the evanescent field formed on the surface is uniform regardless of the length of the nanowire, and thus the optical interactions of the nanowire surface can be easily predicted and utilized.

1. A nanowire-based genetic material inoculation system for delivering a genetic material into a cell, comprising:
a nanowire waveguide;
an optical fiber connected to one end of the nanowire waveguide; and
the genetic material with which a surface of the nanowire waveguide is coated.

2. The nanowire-based genetic material inoculation system of claim 1, wherein the surface of the nanowire waveguide is coated with the genetic material by modifying the surface of the nanowire waveguide by a method selected from the group consisting of a biotin-avidin interaction, a copper-free click chemical reaction, NHS/EDC covalent bonding, and an aldehyde-amine reaction, and attaching the genetic material, linked to a photocleavable linker, to the modified surface of the nanowire waveguide.

3. The nanowire-based genetic material inoculation system of claim 1, wherein the nanowire waveguide is fabricated by bringing a nanopipette, filled with a polyvinyl halide derivative solution containing poly(vinylbenzyl azide) (PVBN₃), polyvinyl halide, or a mixture thereof, into contact with the tip of the optical fiber, and then withdrawing the nanopipette.

4. The nanowire-based genetic material inoculation system of claim 2, wherein the photocleavable linker is a nitrobenzyl-based photocleavable linker.

5. The nanowire-based genetic material inoculation system of claim 1, wherein, when UV light is irradiated into the optical fiber, the UV light reaching the nanowire waveguide forms an evanescent field, so that the genetic material is released into the cell.

6. The nanowire-based genetic material inoculation system of claim 5, wherein the UV light has a wavelength of 300 to 400 nm.

7. The nanowire-based genetic material inoculation system of claim 1, wherein the nanowire waveguide has a diameter of 100 nm to 500 nm.

8. The nanowire-based genetic material inoculation system of claim 1, wherein the genetic material comprises a nucleic acid selected from the group consisting of mRNA, siRNA, miRNA, DNA, and a DNA-RNA duplex.

9. A method for fabricating a nanowire-based genetic material inoculation system, comprising steps of:
fabricating a nanopipette;
fabricating a tapered optical fiber by tapering one end of an optical fiber;
filling the nanopipette with a polyvinyl halide derivative solution containing poly(vinylbenzyl azide) (PVBN₃), polyvinyl halide, or a mixture thereof, and bringing the nanopipette into contact with the tip of the optical fiber;
fabricating a nanowire waveguide by evaporating a solvent from the polyvinyl halide derivative solution while withdrawing the nanopipette in a direction away from the optical fiber; and
coating a surface of the nanowire waveguide with a genetic material by dipping the nanowire waveguide sequentially in a dibenzocyclooctyne-PEG-4-biotin solution and a streptavidin solution, followed by dipping in one selected from the group consisting of a solution containing a biotin-modified genetic material with a photocleavable linker based on biotin-avidin interaction, a solution containing the biotin-modified genetic material without the photocleavable linker, a solution containing a fluorescent substance and the biotin-modified genetic material with the photocleavable linker, and a solution containing the fluorescent substance and the biotin-modified genetic material without the photocleavable linker.

10. The method of claim 9, wherein the genetic material comprises a nucleic acid selected from the group consisting of mRNA, siRNA, miRNA, DNA, and a DNA-RNA duplex.

11. A nanowire-based genetic material inoculation method, comprising steps of:
preparing the nanowire-based genetic material inoculation system of claim 1;
inserting the nanowire waveguide into a cell; and
irradiating UV light through the optical fiber to release the genetic material into the cell.

12. The nanowire-based genetic material inoculation method of claim 11, wherein the UV light has a wavelength of 300 to 400 nm.

13. The nanowire-based genetic material inoculation method of claim 11, wherein, when the UV light is irradiated into the optical fiber, the UV light reaching the nanowire waveguide forms an evanescent field, so that the genetic material is separated from the surface of the nanowire waveguide and released into the cell.

14. The nanowire-based genetic material inoculation method of claim 11, wherein the cell is a single living cell, and the nanowire waveguide passes through a cell membrane, nuclear membrane, or organelle membrane within the cell without killing the cell, and releases the genetic material at a desired location in the intracellular environment.


SEQUENCE LISTING (7 sequences)

SEQ ID NO: 1 - DNA, 18 bp, synthetic construct
  source 1..18: mol_type = other DNA
tttcacccttcactaggt

SEQ ID NO: 2 - DNA, 18 bp, synthetic construct
  source 1..18: mol_type = other DNA
tttcacccttcactaggt

SEQ ID NO: 3 - RNA, 30 nt, synthetic construct
  source 1..30: mol_type = other RNA
tttttgcgtgatcttcaccgacaagatcat

SEQ ID NO: 4 - RNA, 30 nt, synthetic construct
  source 1..30: mol_type = other RNA
tttttcaacaagatgaagagcaccaaaggc

SEQ ID NO: 5 - RNA, 27 nt, synthetic construct
  source 1..27: mol_type = other RNA
atgatcttgtcggtgaagatcacgctg

SEQ ID NO: 6 - RNA, 27 nt, synthetic construct
  source 1..27: mol_type = other RNA
gcctttggtgctcttcatcttgttggt

SEQ ID NO: 7 - DNA, 39 bp, synthetic construct
  source 1..39: mol_type = other DNA
ttttttttttctcctttctctttcttcccttcctttctc